(19) United States
(12) Patent Application Publication
(10) Pub. No.: US 2006/0136037 A1
(43) Pub. Date: Jun. 22, 2006

(54) Small vessel stent designs

(76) Inventors: Nicholas C. DeBeer, Montara, CA (US); Frank P. Becking, Palo Alto, CA (US)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 11/238,646

(22) Filed: Sep. 28, 2005

Related U.S. Application Data
(63) Continuation of application No. 60/619,437, filed on Oct. 14, 2004.

(51) Int. Cl. A61F 2/06

(52) U.S. Cl. 623/1.15

(57) ABSTRACT

Medical device and methods for delivery or implantation of prostheses within hollow body organs and vessels or other luminal anatomy are disclosed. The subject technologies may be used in the treatment of atherosclerosis in stenting procedures.

### CROSS REFERENCE

This filing claims the benefit provisional patent application Ser. No. 60/619,437, entitled “Small Vessel Stent Designs” filed Oct. 14, 2004 the entirety of which is incorporated by reference.

### BACKGROUND

Implants such as stents and occlusive coils have been used in patients for a wide variety of reasons. One of the most common “stenting” procedures is carried out in connection with the treatment of atherosclerosis, a disease which results in a narrowing and stenosis of body lumens, such as the coronary arteries. At the site of the narrowing (i.e., the site of a lesion) a balloon is typically dilatated in an angioplasty procedure to open the vessel. A stent is set in apposition to the interior surface of the lumen in order to help maintain an open passageway. This result may be effected by means of scaffolding support alone or by virtue of the presence of one or more drugs carried by the stent aiding in the prevention of restenosis.

Various stent designs have been developed and used clinically, but self-expandable and balloon-expandable stent systems and their related deployment techniques are now predominant. Examples of self-expandable stents currently in use are the Magic WALLSTENT® stents and Radius stents (Boston Scientific). A commonly used balloon-expandable stent is the Cypher® stent (Cordis Corporation). Additional self-expanding stent background is presented in: “An Overview of Superelastic Stent Design,” Min. Invas Ther & Allied Technol 2002: 9(3/4) 235-246, “A Survey of Stent Designs,” Min. Invas Ther & Allied Technol 2002: 11(4) 137-147, and “Coronary Artery Stents: Design and Biologic Considerations,” Cardiology Special Edition, 2003: 9(2) 9-14, “Clinical and Angiographic Efficacy of a Self-Expanding Stent” Am Heart J 2003: 145(5) 868-874.

Because self-expanding prosthetic devices need not be set over a balloon (as with balloon-expandable designs), self-expanding stent delivery systems can be designed to a relatively smaller outer diameter than their balloon-expandable counterparts. As such, self-expanding stents may be better suited to reach the smallest vasculature or achieve access in more difficult cases.

To realize such benefits, however, there continues to be a need in developing improved stents and stent delivery systems. Problems encountered with known delivery systems include drawbacks ranging from failure to provide means to enable precise placement of the subject prosthetic, to a lack of space efficiency in delivery system design. Space inefficiency in system design prohibits scaling the systems to sizes as small as necessary to enable difficult access or small-vessel procedures (i.e., in tortuous vasculature or vessels having a diameter less than 3 mm, even less than 2 mm).

Even where a delivery system is sized for such use, the stent itself needs to be adapted to reach high compression ratios. While ease of collapsing the stent for loading in the delivery system can be achieved using longer-length struts in a stent design, doing so results in loss of radial force that the stent can withstand or exert when set within a vessel or other hollow body lumen. Designs are needed that allow reaching high compression ratios without unduly compromising the radial force capacity of the stent. In addition, it is important to manage the stress states in order that the stent be sufficiently durable—either in use or simply in loading the system.

Yet another aspect of stent design requiring improvement in regard to small vessel applications arises in terms of stent conformability to the subject anatomy. The ability of a stent to conform to the shape of the target site is of great importance for the purpose of providing even support to a lumen wall and/or radial force thereto. Good stent/wall contact allows for drug delivery (in case of drug-eluting stents), helps avoid thrombosis formation and/or partially obstructing the subject lumen thereby adversely affecting flow therein.

Another consideration pertinent to self-expanding stent designs concerns frictional forces internal to the subject delivery system. Internal forces can be a significant issue with respect to system actuation. Testing by the assignee hereof has clearly demonstrated a loss of motive force available to actuate a distally located restraint when the delivery system is subject to conditions of or simulating tortuous anatomy.

Consequently, it is important to minimize delivery system internal friction in order that the member restraining the stent can be withdrawn from the same without the need for increasingly large input forces that can damage system components. In this regard, it will be of benefit to provide a stent that exerts lower outward radial forces upon full compression and when held in a collapsed configuration within a delivery system. Lower radial stent forces, when collapsed, result in lower static and dynamic frictional forces between the stent and restraining member during withdrawal of the restraining member and upon breakaway between the two.

Aspects of the present invention are suited to offering improvements in each of the areas of space efficient stent and delivery device design, stent conformability and/or delivery system actuation. Realizing such improvements may be especially useful in the context of small-vessel or other body lumen applications. However, the improvement(s) may be useful in a variety of settings. In addition, it is noted that those with skill in the art may appreciate further advantages or benefits of the present invention.

### SUMMARY OF THE INVENTION

The present invention offers a number of stent delivery system and stent-specific designs especially helpful for use in small vessel (or other hollow body region) applications. The stents themselves will generally be self-expanding upon release from a restraint. Thus, full or complete placement of the stent may be achieved solely upon its release from the delivery device. Still, aspects of the invention may be applicable to balloon-expandable stents and their delivery systems.

Together, the stent and a delivery guide provide a stent delivery system. When loaded, the stent is held by the delivery guide in a collapsed configuration with a sheath or distal restraint. The overall character of the delivery guide, including the sheath or restraint, is highly variable. Various options are described as variously set forth in commonly assigned U.S. patent application Ser. Nos. 10/792,657; 10/792,679,10/792,684,10/991,721 or PCT Application No. US 2004/00008909 or 2005/002680, each application being incorporated by reference herein in its entirety. Still other applicable delivery system features that may be applied in such systems are described in Ser. No. 10/967,079 or 11/211,129, each also incorporated by reference in its entirety.

The present invention, however, focuses on the stent to be delivered. Still, an aspect of the invention concerns the stent and delivery guide as an assembly suitable for deploying one or more stents.

Regarding the stent itself, each embodiment is suited for small vessel use by virtue of various features. Many of the stents according to the present invention offer designs for minimizing stent wall thickness. Reduced wall thickness minimizes the space occupied necessarily occupied by the stent in the delivery system. Conserving space is very important in designing delivery systems that are able to access small vessels. As such, stents according to the present invention have a strut thickness-to-strut width ratio around about 1:1, and generally not more than about 3:2.

Achieving high expansion ratios (as elaborated upon below) is also important for producing small vessel or minimized crossing profile delivery systems. Basically, the stent compression ratio that can be achieved determines the outside diameter to which the stent can be compressed, and subsequently be allowed to return to for treating a given vessel size. The strut thickness, then—in turn—sets the internal dimension of the stent and sizing for any delivery device components internal thereto.

As for reducing in-sheath or in-restraint forces, this is an important factor for a number of reasons. For one, lower force requirements for holding a stent in a compressed or deployment configuration plays into system design in terms of material selection, sizing, etc. for the restraining member. Still further, lower compression forces translate to lower normal forces between the stent and sheath or restraint (hereinafter the “tubular member”). Decreasing these forces affects frictional forces/losses in removing the tubular member in a sliding sheath or restraint based approach.

A first variation of the invention addresses issues of achieving high compression ratios as well as reducing in-restraint forces by developing a stent that compacts into an advantageous shape. In a preferred implementation, at least some of the struts form diamond-shaped cells (sets of four interconnected struts or arms/legs). Such a configuration is suitable for thin-walled high compression ratio stents because of the strength offered by the design. Yet, strut features disclosed herein are applicable to “zig-zag” type stents or other patterns. See, “A Survey of Stent Designs” referenced above and incorporated herein by reference in its entirety for further optional patterns as may be employed.

The stent design includes specially-shaped “S” curve struts. The shape of the curve is set so that, when fully compressed, the struts deform to a substantially straight condition. In addition, instead of simply deforming into a “slotted tube” type body, opposing or circumferentially adjacent (rather than neighboring or axially adjacent) the compressed struts form “teardrop” shaped spaces therebetween. In the alternative, the struts themselves may be seen as defining a series of closely-packed teardrop shaped forms (i.e., no intermediate or intervening structure is presented in the array or arrangement of shapes).

As for the negative space profile, however, it is defined at one end by a full radius between the adjacent struts and on another side where the same struts contact or nearly contact. The shape preferably runs about the full length of the strut(s). In other words, any contact or near contact between the adjacent struts preferably occurs across from the adjacent radius at the junction/connection of adjacent struts. In this manner, given substantially even strut width and strut thickness, the dimensions are minimized along the length of the strut thereby concentrating bending to the full extent possible in the medial section of the struts as opposed to the higher stress end regions.

Still, contact may be made between adjacent struts earlier, effectively shortening the teardrop shape. The degree to which the contact point moves inward from the strut ends may vary. Yet, the struts are preferably designed so that no other-wise shaped gaps are present. In other words, the stent is preferably designed to compact fully except in the teardrop shaped areas left intentionally open for stress reduction at the strut junction bends. While other designs incorporate teardrop shaped sections—see, e.g., U.S. Pat. No. 6,533,807—they have bent struts where those bends introduce their own stress concentration points. In contrast, the struts employed in the present invention are intended to be curvilinear in an uncompressed state and compress to a substantially straight or at least a smooth profile devoid of stress raising features along the struts. In this manner, it is believed that maximum compaction and expansion ratios and/or lower stress stent designs are provided by this variation of the present invention.

In addition, when the struts are compressed as described, and the stent reaches its minimum diameter but the struts do not contact, or have minimal contact as desired, the body has a better chance of maintaining a cylindrical profile. In comparison, where strut members are configured such that substantial contact is expected upon full compression such as in the '807 patent or otherwise (especially when they have rounded edges as common to electropolished prostheses), they will tend to ride up over one another. Even when they do not, the propensity to do so will result in additional forces normal to the surface of the compressed stent that can increase engagement with an overriding sheath. In addition, deformation of the sheath material can even result in a positive interlock between the parts as portions of the stent protrude outward. Such conditions are avoided by the aforementioned stent according to the present invention.

In theory, producing a stent able to achieve minimal strut contact in a compressed state should not be difficult. Such stents have been produced by cutting slits in a tube to define the cells such as by laser machining, expanding the stent, and then heat-setting the shape of the stent in an expanded configuration. Such a stent should then be compressed down to a shape that resembles its original “slofted tube” form. Unfortunately, material so-treated looses elasticity in view of the material processing steps. In addition, irregularity of the stent pattern derived from expansion of tubing is a concern. These effects are well documented. See, “NITINOL Tubular Stents: Comparing Two Manufacturing Methods”: SMST proceeding of the First European conference on Shape Memory and Superelastic Technologies, 1999 (165-170).

Accordingly, a stent that is designed and produced at an initially expanded geometry (rather than a post-formation expanded and heat set geometry) will offer superior material performance and actually have a different physical character than one produced by expansion and heat setting. Still further, it is not possible or at least highly infeasible to cut stress-relief features for a final stent shape into a very small diameter tube to be expanded (e.g., on the order less than about 0.020 to about 0.012 inches or smaller) in view of current laser beam widths and available power at the reduced beam widths. A lack of stress relief features limits the compression ratios that can be achieved before material cracking and breakage. Of course, stress relief features could in some such cases be etched into the material. However, such a result is accomplished only at increased cost and without alleviating the issue of expansion and heat setting noted above.

In addition, beam width limitations do not allow for achieving the tear-drop shaped profile originally in cut tubing as noted above where the end of the stent struts are touching or nearly-so. Moreover, limitations in etching techniques in which etching width and depth far exceeds a 1:1 ratio for such a section (namely, it would be on the order of at least about 1:2 or more preferably about 1:5 to about 1:10 to about 1:20 or more) would yield—at best—highly irregular and/or undercut strut geometry.

In any case, where a stent is cut in a fully-expanded or nearly fully-expanded shape (such that further expansion will not result in the noted problems) according to the present invention, both the noted stress-relief and close-proximity features are easily (and cost-effectively) incorporated in the design. The present invention contemplates a number of different approaches for such stent production.

In one approach, a precursor stent design is provided out of the material (composition, thickness, etc.) intended for use in the final stent design. The design is then expanded. Such activity may occur by a physical process as described above by way of providing a slit-tube stent that is then forced open. Whether the shape is heat set or not, the geometry observed will then be used to generate or adopted directly as the final cut pattern intend for the stent to be cut at full size.

Naturally, it would be preferred that the precursor stent include stress relief features as will be incorporated into the final stent design. However, if the stent tubing is too small to provide such features, then a larger scale model of the final stent design may be employed or such features may simply be incorporated in the final design.

In the alternative, the design process may proceed based on a purely computational model or approach, where “expanding” the stent is performed on an exemplary strut or an entire stent through Finite Element Analysis (FEA). The resulting expanded shape will then be adopted as the form for struts in which to originally cut the subject stent.

Such data processing aspects of the invention can be implemented in digital electronic circuitry, or in computer hardware, firmware, software, or in combinations of thereof. Data processing aspects of the invention can be implemented in a computer program product tangibly embodied in a machine-readable storage device for execution by a programmable processor; and data processing method steps of the invention can be performed by a programmable processor executing a program of instructions to perform functions of the invention by operating on input data and generating output.

In one implementation of the subject invention, the FEA program Abaqus™ was run on a desktop computer to generate an output data set used to produce a final stent design. The program has proven highly accurate in the past to model stent geometry and generated stress-strain rates. Indeed, such modeling is required in order to form any reasonable estimation or opinion as to the stresses and strains generated in such a superelastic NITINOL part. It is a common observation that the non-linear character of superelastic NITINOL results in behavior that is quite complex.

As for the particular analysis, a desirable precursor shape for a collapsed-shape strut was entered into the program by conventional means along with the material properties for the NITINOL alloy used (superelastic NiTi: 54.5 to 57% Ni, balance Ti, and other typical agents). Next, the program was employed to force the strut to an open configuration. In expanding the strut, it assumed a stress state and shape yielding the S-curve noted above. Output file data was then imported into the Computer Aided Design (CAD) program SolidWorks™ and used to generate a strut pattern suitable for machining.

According to the present invention, the output data set provided by the computational approach to stent or stent strut expansion, may simply be displayed or printed. However, for the sake of convenience and use in other applications, the data set is preferably physically stored on a computer readable medium (such as EPROM, EEPROM, flash memory devices; magnetic disks such as internal hard disks and removable disks; magneto-optical disks; and CD or DVD disks, etc.) from which the data can be retrieved and/or manipulated. In the latter case, such manipulation may be with another computer program serving a different function to finalize the subject stent design. As such, one aspect of this variation of the invention provides for an initial data set representing a stent in a desired compressed configuration. This data set, representing a stent precursor design is then manipulated to produce either an intermediate or final digital data set. The final data set may comprise coordinates or output files needed to machine or cut a stent. Similarly, the data set may take the form of a print or drawing describing the stent to be produced, or even some form of mathematical parameterization of the final stent design.

In any case, another aspect of the present invention involves the use of a special mode of stent construction advantageously suited for use with a NiTi alloy not previously used in stent production. In fact, the particular NiTi alloy that may be employed in this stent construction has not been available in the form of tubing, and according to a vendor of the material (Furukawa Techno Material Co., Ltd.), the material, identified by its only vendor as FHP-NT, cannot be produced in tubing. As such, the process employed may offer the option of making stents not here-to-fore possible—or at least impractical.

The referenced material has been offered for use as a material that is able to reach superelastic-type stress levels in the production of medical guidewires. The material is described in Furukawa literature as having no yield point, showing no super-elastic plateau and having small residual strain after 4% strain. Further, it is purported to show stable characteristics at any temperature such that its physical properties do not change according to thermal environmental conditions. The alloy is 54-57 wt % Ni—Ti displaying typical properties of 1270 MPa stress at 4% strain, 800 MPa Stress Hysterisis at 2% strain, and 0.05% residual strain after 4% strain. In comparison, a more typical superelastic alloy displays 490 MPa stress at 4% strain, 265 MPa Stress Hysterisis at 2% strain, and 0% residual strain after 4% strain.

An aspect of the present invention involves the use of this material or one with similar performance such as the cold-worked Ni—Ti alloy described in U.S. Pat. No. 6,602,272 (incorporated herein by reference in its entirety, and specifically for its teaching regarding alloy processing in which the material may be cold formed and further cold worked below the recrystallization temperature of the material) that offers reversible superelastic level strain rates, but has little or substantially no “plateau” region (i.e., a typical “flag” shaped superelastic stress-strain curve). Without the plateau region, it can be said that the material does not exhibit traditional NITINOL “pseudoelastic” behavior. Alternatively, the material may actually be referred to as exhibiting “linear pseudoelastic” behavior without a phase transformation or onset of stress-induced martensite. The subject approaches to stent construction with the material, as well as a stent otherwise produced from such material form aspects of the present invention.

It is noted that without the material stress level “plateau”, a stent constructed of the material may be the case in some instances require higher hold-down forces. However, such a stent can be designed to operate in a vessel or other hollow body conduit at greater compression (both in terms of percentage from unconstrained and/or overall force) without concern of collapse upon reaching a point where a small amount of additional force will drive large-scale compression. In the alternative, it may be possible to design a stent to deliver comparable in-vessel radial forces as a typical NITIONOL stent, while using less material. Still further, with a stent that requires greater compression to generate the same forces, the setup may be more forgiving of tapered vessel anatomy because the forces generated will be relatively more evenly distributed despite unequal compression over the length of the stent.

One approach to stent construction that is advantageously employed in producing an FHP-NT or similar material (i.e., an FHP-NT type material) stent involves braiding or winding a stent into a pattern with known techniques and then spot welding—the crossed-over material where it overlaps. In order to reach higher radial force capacities in higher compression ratio designs, especially for use in small vessels, the stent pattern will often comprise a plurality of closed cells. Often, the type of welding employed will be friction welding. Especially for small stent designs, such an approach offers advantages because the resultant product will have thinner (i.e., not double-thick) junctions as would otherwise result from joining fully overlapped members. Still, other welding techniques such as laser welding may be employed as well as brazing techniques, etc. to join the material.

However the connections are formed, the structure will advantageously be laser cut or electrical discharge machined (EDM) to improve the design by adding stress relief features in order to enable the stent to reach higher compression ratios. Otherwise, the device will be susceptible to cracking or breakage upon compression, or likely to respond with uneven compression forces complicating delivery system loading.

In addition to junction modification, it may also be desired to modify the strut geometry. Still further, the structure's cells may be opened or the pattern otherwise modified as desired. In other words, the approach to stent construction is not limited to producing only closed-cell stent geometries, nor is the construction approach limited to use with FHP-NT type materials. The construction approach may be advantageously employed in connection with other materials not produced (or easily provided) in the form of tubing. In any case, the starting point for construction is typically a welded, brazed or soldered wire or ribbon structure that is later modified.

Regardless, the aforementioned approaches to stent construction will most advantageously be employed when it is desired to produce a stent having a width-to-thickness ratio of about 1:1 or greater (i.e., a ratio of 3:2, 2:1, etc). In instances where material having an appreciably smaller width to thickness ratio is to be employed (e.g., about 1:2, 1:3 or up to 1:10, etc.), the teachings of U.S. Pat. No. 6,454,795 are employed in constructing a stent of FHP-NT or like material. Because the stent configuration in the '795 patent is capable of reaching high expansion ratios, and using FHP-NT type offers improved radial force capacity and/or operating range, such a stent will in many instances be suitable for small vessel use.

While simply forming a stent of shaped and welded or soldered wire is known, the use of FHP-NT type material in such a stent offers previously unavailable characteristics—regardless of the stent construction. As variously stated above, self-expanding stents produced with this material can offer a significantly higher radial force (for a given strut size/weight). In the alternative, the stents can be made with less material, while offering comparable radial force (upon deployment) characteristics as other known stents. The selection of FHP-NT type material, used outside of the guidewire setting for which it was designed, together with the geometry clean-up procedures contemplated is believed to offer a new class of self-expanding stents.

Such a stent is able to be highly compressed, up to about 7% or 8% strain like typical NITINOL, and yet offer higher deployed stresses by enabling in-situ strain rates over 1.5% (i.e., into a range where other superelastic NiTi stents undergo a martinsitic phase change at body temperature, thereby limiting radial force potential and implicating fatigue issues).

Consequently, stents according to this aspect of the present invention can be oversized to a greater degree than known stents. That is to say, for emplacement within a vessel of a first diameter, the stent in a completely uncompressed state will have a second larger diameter. The radial force exerted by the stent against the vessel wall will be a function of the difference of these diameters, where the vessel diameter limits stent expansion within the vessel.

An FHP-NT type or like stent can be used such that the oversizing relative to the anatomical structure in which it is implanted is up to about 50%, more preferably up to about 33% or at least over 25%—which is a common upper limit for oversizing known self-expanding stents. Accordingly, such a stent may be emplaced in a 3.5 mm vessel or other hollow body structure with an oversize between about 0.5 and about 1.5 mm diameter; between about 0.5 and about 1.25 mm diameter oversizing for a 3.0 mm diameter vessel; between about 0.5 and about 1.0 mm oversizing for a 2.5 mm diameter vessel; and so-forth.

In any case, by employing any of a number of the referenced features (alone or in combination), stents/implants and delivery guides offering desirable functionality according to the present invention are amenable to scaling to sizes and offering functionality not previously achieved. Consequently, the systems may be used in lieu of a guidewire, such as in a “guidewireless” delivery approach. Still further, rather than providing an “over-the-wire” delivery system as referenced above, the present systems may be regarded as “on-the-wire” delivery systems, since—in effect—delivery is accomplished by a system in which the stent is carried by a delivery guide occupying a catheter lumen that would commonly otherwise be used to accommodate a guidewire.

Whether used in such a manner or otherwise (such as by configuring the subject systems for treating larger peripheral vessels), the present invention includes systems comprising any combination of the features described herein. Methodology described in association with the devices disclosed also forms part of the invention. Such methodology may include that associated with completing an angioplasty, bridging an aneurysm, deploying radially-expandable anchors for pacing leads or an embolic filter, or placement of a prosthesis within neurovasculature, an organ selected from the kidney and liver, within reproductive anatomy such as the vasdeferens and fallopian tubes or for other applications.

### DEFINITIONS

The term “stent” as used herein includes any stent, such as coronary artery stents, other vascular prosthesis, or other radially expanding or expandable prosthesis or scaffold-type implant suitable for the noted treatments or otherwise. Exemplary structures include wire mesh or lattice patterns and coils, though others may be employed in the present invention. The “diameter” of the stent need not be circular—it may be of any open configuration.

A “self-expanding” stent as used herein is a scaffold-type structure (serving any of a number of purposes) that expands from a reduced-diameter (be it circular or otherwise) configuration to an increased-diameter configuration. The mechanism for shape recover may be elastic, pseudoelastic or as otherwise described herein. As such, suitable self expanding stent materials for use in the subject invention include Nickel-Titanium (i.e., NiTi) alloy (e.g., NITINOL) and various other alloys or polymers. Certain self-expanding materials are, however, specific to certain aspects of the present invention—particularly superelastic NiTi and FHP-NT or other cold-worked NiTi alloys.

### BRIEF DESCRIPTION OF THE DRAWINGS

Each of the figures diagrammatically illustrates aspects of the invention. Of these:

FIG. 1 shows a heart in which its vessels may be the subject of one or more angioplasty and stenting procedures;

FIG. 2A shows an expanded stent cut pattern as may be used in producing a stent according to a first aspect of the invention; FIG. 2B shows an enlarged detail of the stent cut pattern in FIG. 2A;

FIGS. 3A-3L illustrate stent deployment methodology to be carried out with the subject delivery guide member;

FIG. 4 provides an overview of a delivery system incorporating at least one of the subject stents;

FIG. 5A is a plan view of a stent strut as employed in the stent cut pattern of FIGS. 2A and 2B, but in a compressed state; FIG. 5B is an enlarged view of the highlighted end of the strut shown in FIG. 5A; FIG. 5C shows a section of a compressed stent assembled from strut sections as shown in FIG. 5A;

FIGS. 6A and 6B are stress-strain curves illustrating typical NITINOL performance and FHP-NT alloy performance, respectively;

FIG. 7A shows a plan view of a welded strut junction and may be employed in providing and FHP-NT stent; FIG. 7B shows the same strut junction in phantom line with a new machined profile in solid line illustrating a final stent junction geometry; and

FIG. 8A is a perspective view of another type of stent possibly employing FHP-NT stent construction; FIG. 8B is a detailed plan view of the manner in which the stents constituent parts are connected.

In the figures, like elements in some cases are indicated by a related numbering scheme. Furthermore, variation of the invention from the embodiments pictured is, of course, contemplated.

### DETAILED DESCRIPTION OF THE INVENTION

Various exemplary embodiments of the invention are described below. Reference is made to these examples in a non-limiting sense. They are provided to illustrate more broadly applicable aspects of the present invention. Various changes may be made to the invention described and equivalents may be substituted without departing from the true spirit and scope of the invention. In addition, many modifications may be made to adapt a particular situation, material, composition of matter, process, process act(s) or step(s) to the objective(s), spirit or scope of the present invention. All such modifications are intended to be within the scope of the claims made herein.

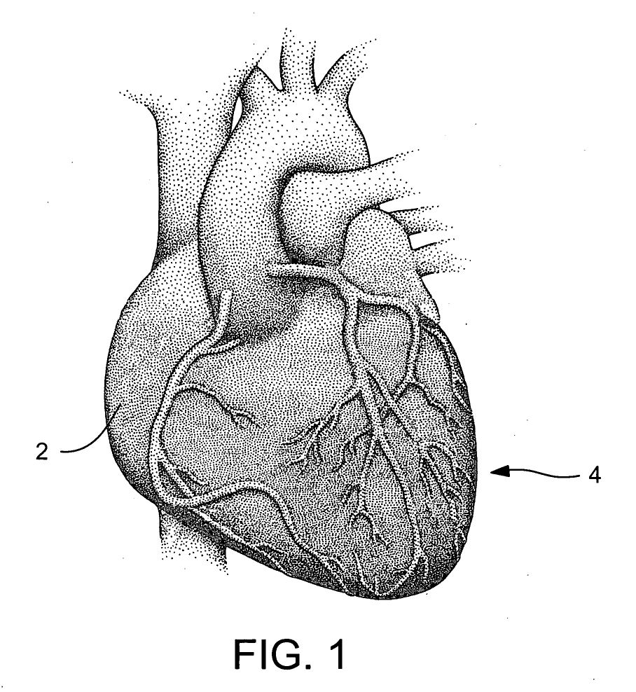

In light of this framework, FIG. 1 shows a heart 2 in which its vessels may be the subject of one or more angioplasty and/or stenting procedures. To date, however, significant difficulty or impossibility is confronted in reaching smaller coronary arteries 4. If a stent and a delivery system could be provided for accessing such small vessels and other difficult anatomy, an additional 20 to 25% of coronary percutaneous procedures could be performed with such a system. Such a potential offers opportunity for huge gains in human healthcare and a concomitant market opportunity in the realm of roughly $1 billion U.S. dollars—with the further benefit of avoiding loss of income and productivity of those treated.

Features of the present invention are uniquely suited for a system able to reach small vessels (though use of the subject systems s not limited to such a setting.) By “small” coronary vessels, it is meant vessels having an inside diameter between about 1.5 or 2 and about 3 mm. These vessels include, but are not limited to, the Posterior Descending Artery (PDA), Obtuse Marginal (OM) and small diagonals. Conditions such as diffuse stenosis and diabetes produce conditions that represent other access and delivery challenges which can be addressed with a delivery system according to the present invention. Other extended treatment areas addressable with the subject systems include vessel bifurcations, chronic total occlusions (CTOs), and prevention procedures (such as in stenting of vulnerable plaque).

Assuming a means of delivering one or more appropriately-sized stents, it may be preferred to use a drug eluting stent in such an application to aid in preventing restenosis. A review of suitable drug coatings and available vendors is presented in “DES Overview: Agents, release mechanism, and stent platform” a presentation by Campbell Rogers, Md. incorporated by reference in its entirety. However, bare-metal stents may be employed in the present invention.

While some might argue that the particular role and optimal usage of self expanding stents has yet to be defined, they offer an inherent advantage over balloon expandable stents. The latter type of devices produce “skid mark” trauma (at least when delivered uncovered upon a balloon) and are associated with a higher risk of end dissection or barotraumas caused at least in part by high balloon pressures and related forces when deforming a balloon-expandable stent for deployment.

Yet, with an appropriate deployment system, self-expanding stents may offer one or more of the following advantages over balloon-expandable models: 1) greater accessibility to distal, tortuous and small vessel anatomy—by virtue of decreasing crossing diameter and increasing compliance relative to a system requiring a deployment balloon, 2) sequentially controlled or “gentle” device deployment, 3) use with low balloon pre-dilatation (if desirable) to reduce barotraumas, 4) strut thickness reduction in some cases reducing the amount of “foreign body” material in a vessel or other body conduit, 5) opportunity to treat neurovasculature—due to smaller crossing diameters and/or gentle delivery options, 6) the ability to easily scale-up a successful treatment system to treat larger vessels or vice versa, 7) a decrease in system complexity, offering potential advantages both in terms of reliability and system cost, 8) reducing intimal hyperplasia, and 9) conforming to tapering anatomy—without imparting complimentary geometry to the stent (though this option exists as well).

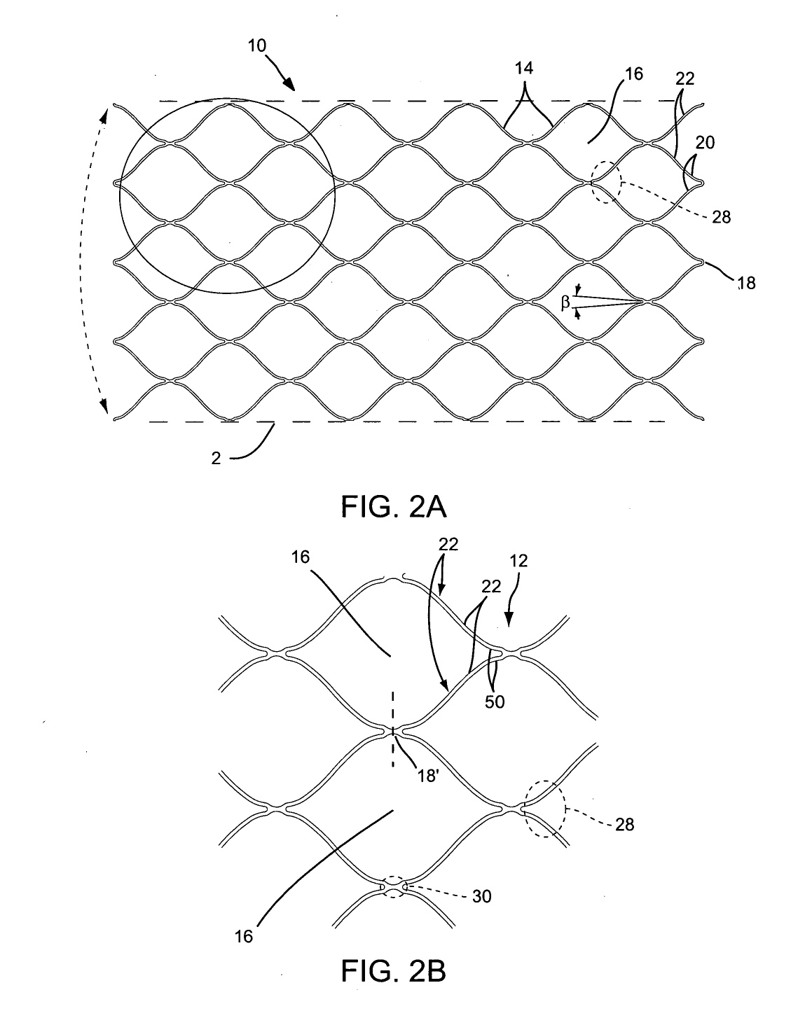

At least some of these noted advantages may be realized using a stent 10 as shown in FIG. 2A. The stent pattern pictured is well suited for use in small vessels. It may be collapsed to fit in a delivery system with an outer diameter of about 0.018 inch (0.46 mm), or even smaller to about 0.014 inch (0.36 mm)—including the restraint/joint used to hold it down—and expand to a size (fully unrestrained) between about 1.5 mm (0.059 inch) or 2 mm (0.079 inch) or 3 mm (0.12 inch) and about 3.5 mm (0.14 inch). Given the thickness of any restraining features and stent coatings, the stent itself may have an outer diameter between about 0.001 and about 0.005 smaller than the outer diameter of the delivery system within this size range.

In use, the stent will be sized so that it is not fully expanded when fully deployed against the wall of a vessel in order to provide a measure of radial force thereto (i.e., the stent will be “oversized” as discussed above). The force will secure the stent and offer potential benefits in reducing intimal hyperplasia and vessel collapse or even pinning dissected tissue in apposition.

Stent 10 preferably comprises NiTi that is superelastic at or below room temperature and above (i.e., as in having an A_{f} as low as 15 degrees C. or even 0 degrees C.). Also, the stent is preferably electropolished. The stent may be a drug eluting stent (DES). Such drug can be directly applied to the stent surface(s), or introduced into an appropriate matrix set over at least an outer portion of the stent. It may be coated with gold and/or platinum to provide improved radiopacity for viewing under medical imaging.

In a 0.014 inch delivery system (one in which the maximum nominal outer diameter of the stent/coating and guide member/restraint have a diameter that does not exceed 0.014 inch), the thickness of the NiTi is about 0.0025 inch (0.64 mm) for a stent adapted to expand to 3.5 mm in a free state. Such a stent is designed for use in a 3 mm vessel or other body conduit, thereby providing the desired radial force in the manner noted above. Further information regarding radial force parameters in coronary stents may be noted in the article, “Radial Force of Coronary Stents: A Comparative Analysis,” Catheterization and Cardiovascular Interventions 46: 380-391 (1999), incorporated by reference herein in its entirety.

In one manner of production, the stent in FIG. 2A is laser or EDM cut from round NiTi tubing, with the flattened-out pattern shown wrapping around the tube as indicated by the double-arrow line. In such a procedure, the stent is preferably cut in its fully-expanded shape. By initially producing the stent to full size, the approach allows cutting finer details in comparison to simply cutting a smaller tube with slits and then heat-expanding/annealing it into its final (working) diameter. Avoiding post-cutting heat forming also reduces production cost as well as the above-reference effects.

Regarding the finer details of the subject stent, as readily observed in the detail view provided in FIG. 2B, necked down bridge sections 12 are provided between axially/horizontally adjacent struts or arms/legs 14, wherein the struts define a lattice of closed cells 16. Terminal ends 18 of the cells are preferably rounded-off so as to be atraumatic.

To increase stent conformability to tortuous anatomy, the bridge sections can be strategically separated or opened as indicated by the broken line in FIG. 2B. To facilitate such tuning of the stent, the bridge sections are sufficiently long so that fully rounded ends 18′ may be formed internally to the lattice just as shown on the outside of the stent if the connection(s) is/are severed to separate adjacent cells 16. Whether provided as ends 18 or adjoined by a bridge section 12, junction sections 28 connect circumferentially or vertically adjacent struts as illustrated. Where no bridge sections are provided, the junction sections can be unified between horizontally adjacent stent struts as indicated in region 30.

The advantage of the optional double-concave profile of each strut bridge 12, however, is that it reduces material width (relative to what would otherwise be presented by a parallel side profile) to improve flexibility and thus trackability and conformability of the stent within the subject anatomy while still maintaining the option for separating/breaking the cells apart.

Further optional features of stent 10 are employed in the cell end regions 18 of the design. Specifically, strut ends 20 increase in width relative to medial strut portions 22. Such a configuration distributes bending (during collapse of the stent) preferentially toward the mid region of the struts. For a given stent diameter and deflection, longer struts allow for lower stresses within the stent (and, hence, a possibility of higher compression ratios). Shorter struts allow for greater radial force (and concomitant resistance to a radially applied load) upon deployment.

Naturally, the overall dimensions of the cells and indeed the number of cells provided to define axial length or diameter may be varied (as indicated by the vertical and horizontal section lines in FIG. 2A). What is consistent, however, is that the “S” curve defined by the struts is produced so that the stent will reach a desired compressed shape. Exemplary means of producing the shape are summarized above.

However derived, the strut shape provided is such that the stent can be compressed or collapsed under force to provide an advantageous strut—and indeed, stent—profile. By utilizing a stent design that minimizes problematic strain (even one that uses the same to provide an improved compressed profile), very high compression ratios of the stent may be easily achieved. Compression ratios (from a fully expanded outside diameter to a fully compressed outside diameter—expressed in those terms used by physicians) of as much as 3.5 mm 0.014 inch (about 10×) or more are possible—with or without a drug coating and/or restraint used. Compression ratios of 3.0 mm: 0.014 inch (about 8.5×), 3.5 mm: 0.018 inch (about 7.5×), 3.0 mm: 0.018 inch (about 6.5×), 2.5 mm: 0.014 inch (about 7×), 2.5 mm: 0.018 inch (about 5.5×), 2.0 mm: 0.014 inch (about 5.5×), 2.0 mm: 0.018 inch (about 4.5×) offer utility not heretofore possible with existing systems as well.

These selected sizings (and expansion ratios) correspond to treating 1.5 to 3.0 mm vessels by way of delivery systems adapted to pass through existing balloon catheter and microcatheter guidewire lumens. In other words, the 0.014 inch and 0.018 inch systems are designed to correspond to common guidewire sizes. The system may also be scaled to other common guidewire sizes (e.g., 0.22 inch/0.56 mm or 0.025 inch/0.64 mm) while offering advantages over known systems. Of course, intermediate sizes may be employed as well, especially for full-custom systems. Still further, it is contemplated that the system sizing may be set to correspond to French (FR) sizing. In that case, contemplated system sizes range at least from 1 to 1.5 FR, whereas the smallest known balloon-expandable stent delivery systems are in the size range of about 3 to about 4 FR.

At least when produced at the smallest sizes (whether in an even/standard guidewire or FR size, or otherwise), the system enables a substantially new mode of stent deployment in which delivery is achieved through an angioplasty balloon catheter or small microcatheter lumen. Further discussion and details of “through the lumen” delivery is presented in U.S. patent application Ser. No. 10/746,455 “Balloon Catheter Lumen Based Stent Delivery Systems” filed on Dec. 24, 2003 and its PCT counterpart US2004/008909 filed on Mar. 23, 2004, each incorporated by reference in its entirety.

In “small vessel” cases or applications (where the vessel to be treated has a diameter up to about 3.0 mm), it may also be advantageous to employ a stent delivery system sized at between about 0.022 to about 0.025 inch in diameter. Such a system can be used with catheters compatible with 0.022 inch diameter guidewires. While such a system may not be suitable for reaching the very smallest vessels, this variation of the invention is quite advantageous in comparison to known systems in reaching the larger of the small vessels (i.e., those having a diameter of about 2.5 mm or larger). By way of comparison, among the smallest known over-the-guidewire delivery systems are the Micro-Driver™ and Pixel™ systems by Guidant. These are adapted to treat vessels between 2 and 2.75 mm, the latter system having a crossing profile of 0.036 inches (0.91 mm). A system described in U.S. Patent Publication No. 2002/0147491 for treating small vessels is purported to be capable of being made as small as 0.026 inch (0.66 mm) in diameter.

With respect to such systems, however, it must be appreciated that a further decrease in stent size may be practically impossible in view of material limitations and functional parameters of the stent. Instead, the present invention offers a different paradigm for delivery devices and stents that are scalable to the sizes noted herein.

By virtue of the approaches taught herein, it is feasible to design delivery system diameters to match (or at least nearly match) common guidewire size diameters (i.e., 0.014, 0.018 and 0.022 inch) for small vessel delivery applications. As noted above, doing so facilitates use of the subject stents with compatible catheters and opens the possibility for methodology employing the same as elaborated upon below and in the above-referenced “Balloon Catheter Lumen Based Stent Delivery Systems” patent application.

Of further note, it may be desired to design a variation of the subject system for use in deploying stents in larger, peripheral vessels, biliary ducts or other hollow body organs. Such applications involve a stent being emplaced in a region having a diameter from about 3.5 to about 13 mm (0.5 inch). In this regard, the scalability of the present system, again, allows for creating a system adapted for such use that is designed around a common wire size. Namely, a 0.035 to 0.039 inch (3 FR) diameter crossing profile system is advantageously provided in which the stent expands (unconstrained) to a size between about roughly 0.5 mm and about 1.0 mm greater than the vessel or hollow body organ to be treated. Sufficient stent expansion is easily achieved with the exemplary stent pattern shown in FIGS. 2A and 2B.

Again, as a matter of comparison, the smallest delivery systems known to applicants for stent delivery in treating such larger-diameter vessels or biliary ducts is a 6 FR system (nominal 0.084 inch outer diameter), which is suited for use in an 8 FR guiding catheter. Thus, even in the larger sizes, the present invention affords opportunities not heretofore possible in achieving delivery systems in the size range of a commonly used guidewire, with the concomitant advantages discussed herein in view of the large expansion ratios possible.

Several known stent delivery systems are compatible with (i.e., may be delivered over) common-sized guides wires ranging from 0.014 inch to 0.035 inch (0.89 mm). Yet, none of the delivery systems are themselves known to be so-sized.

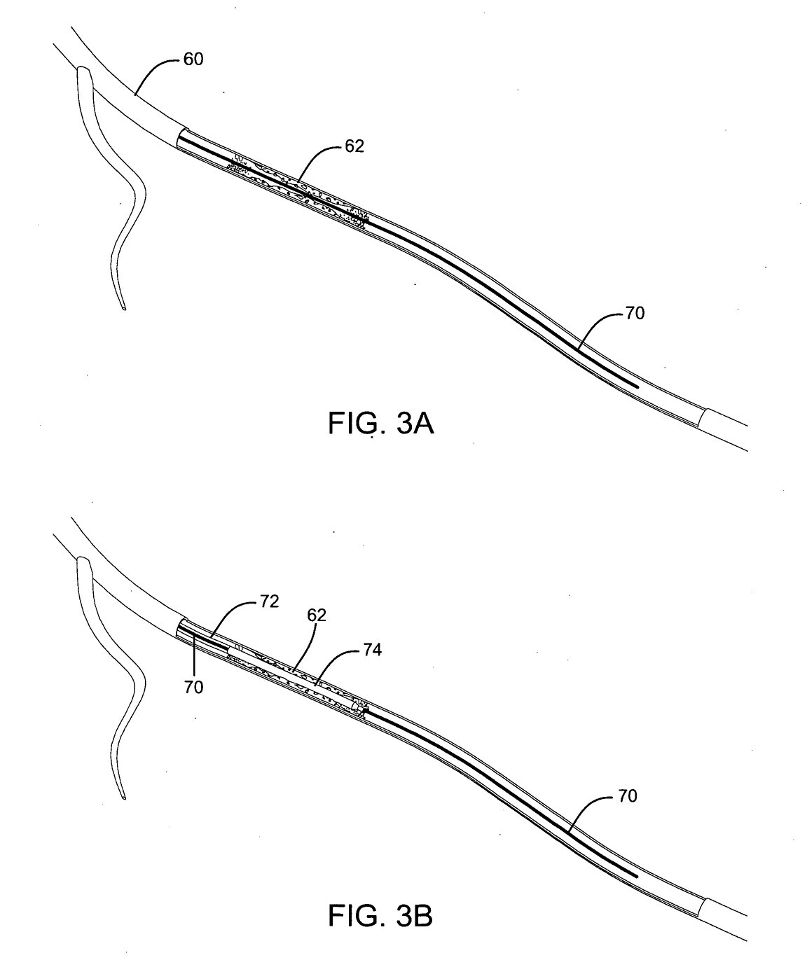

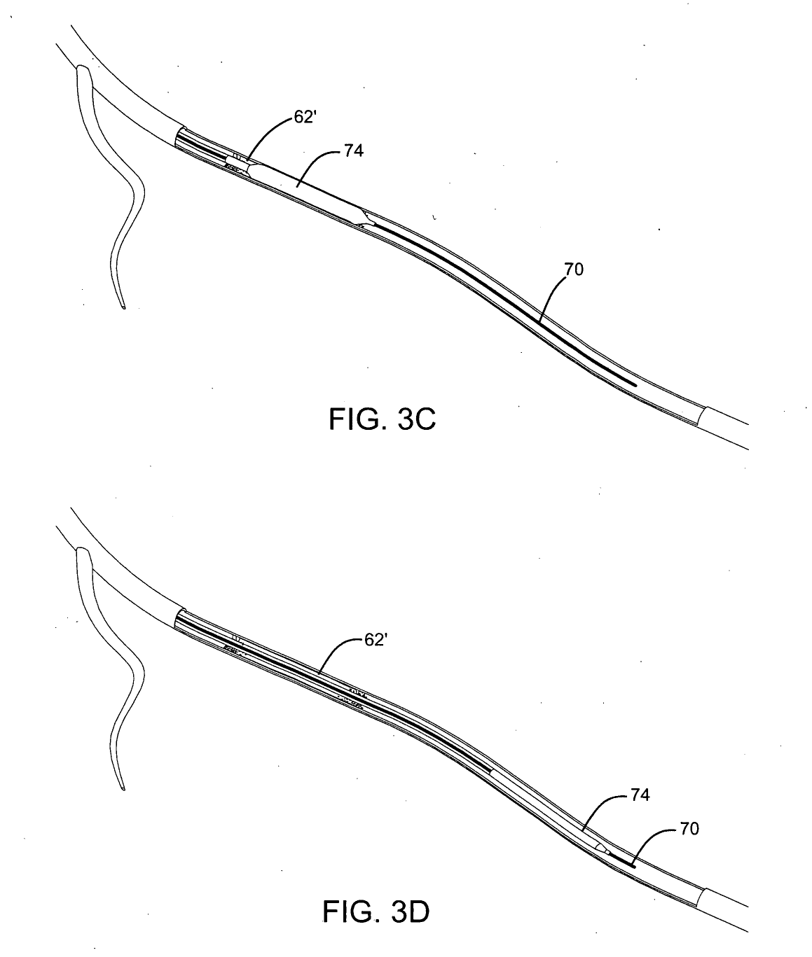

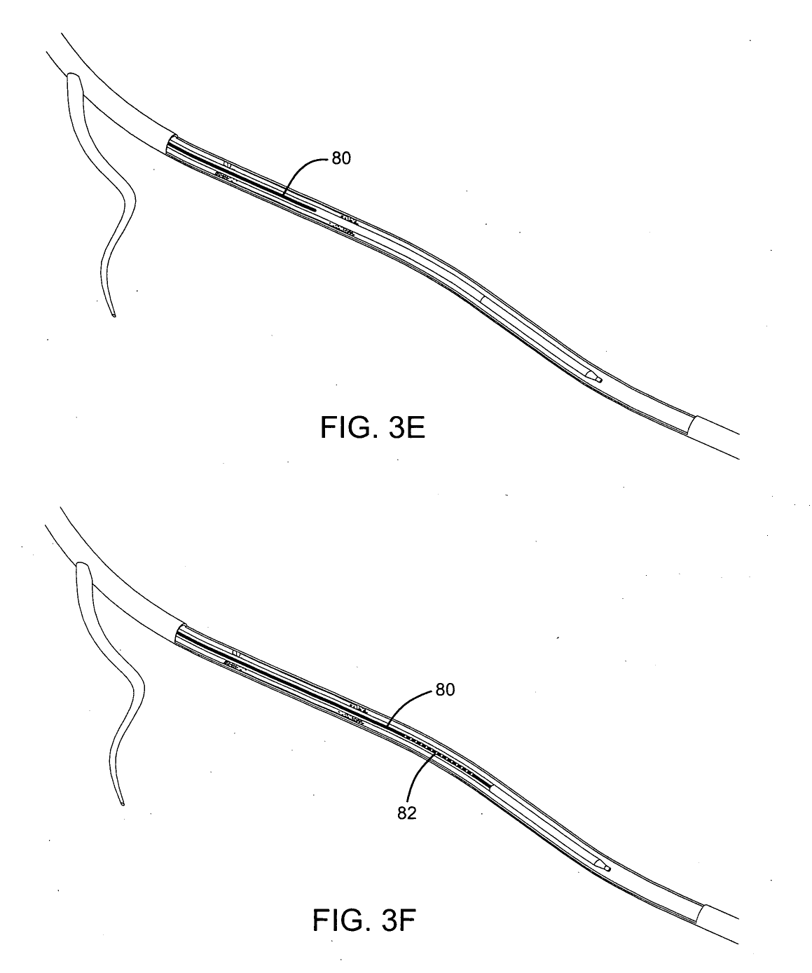

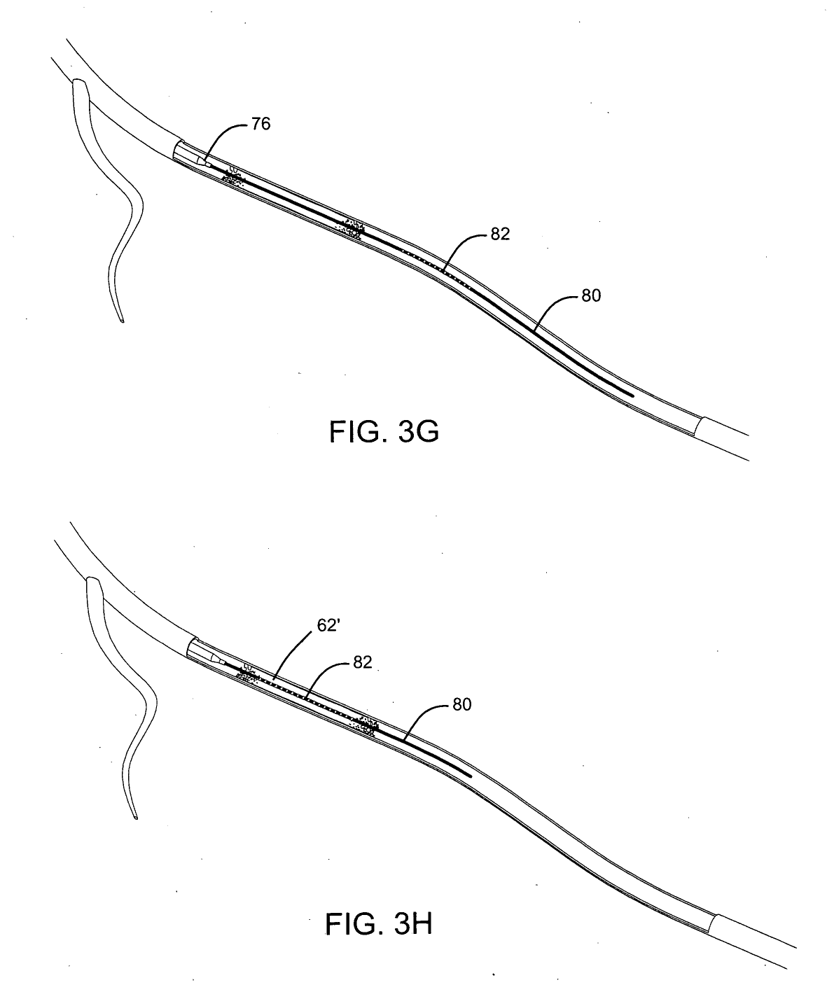

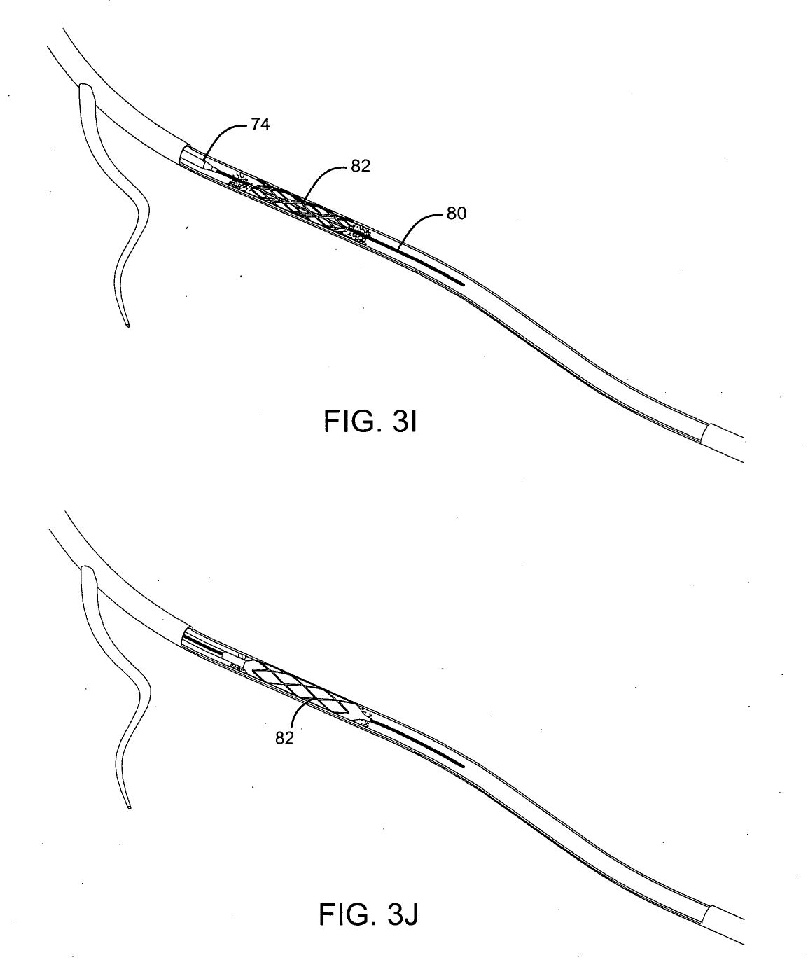

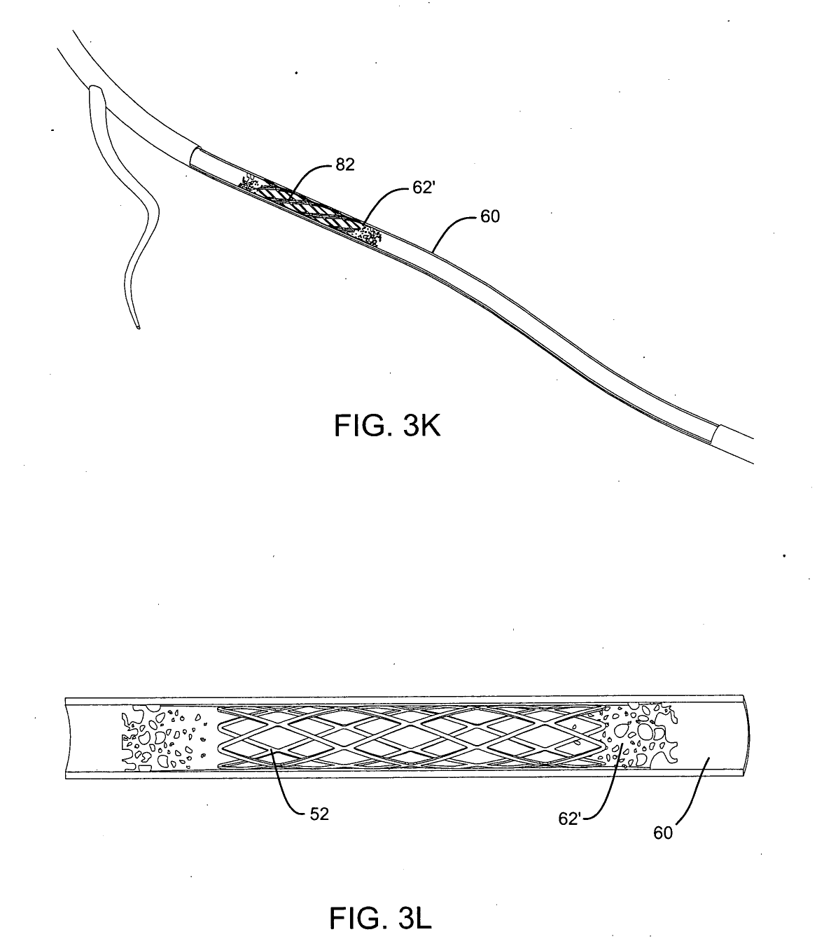

As for the manner of using the inventive system as optionally configured, FIGS. 3A-3L illustrate an exemplary angioplasty procedure. Still, the delivery systems and stents or implants described herein may be used otherwise—especially as specifically referenced herein.

Turning to FIG. 3A, it shows a coronary artery 60 that is partially or totally occluded by plaque at a treatment site/lesion 62. Into this vessel, a guidewire 70 is passed distal to the treatment site. In FIG. 3B, a balloon catheter 72 with a balloon tip 74 is passed over the guidewire, aligning the balloon portion with the lesion (the balloon catheter shaft proximal to the balloon is shown in cross section with guidewire 70 therein).

As illustrated in FIG. 3C, balloon 74 is expanded (dilatated or dialated) in performing an angioplasty procedure, opening the vessel in the region of lesion 62. The balloon expansion may be regarded as “predilatation” in the sense that it will be followed by stent placement (and optionally) a “postdilataton” balloon expansion procedure.

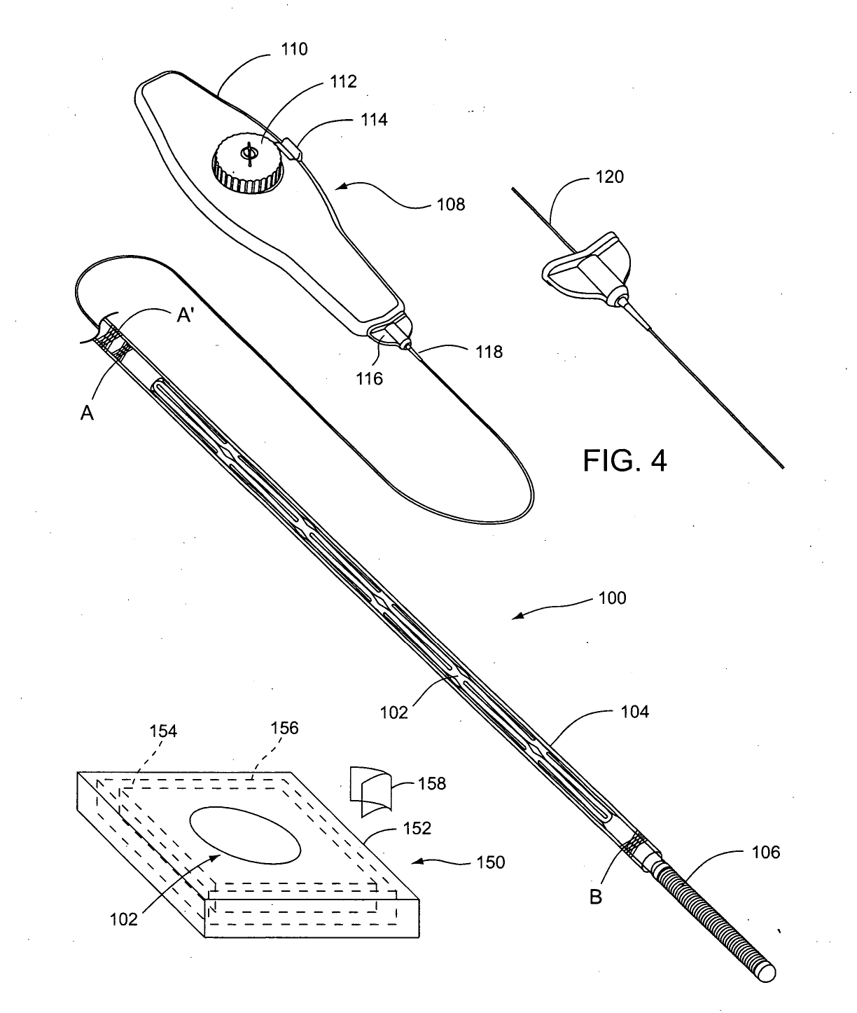

Next, the balloon is at least partially deflated and passed forward, beyond the dilate segment 62′ as shown in FIG. 3D. At this point, guidewire 70 is removed as illustrated in FIG. 4E. It is exchanged for a delivery guide member 80 carrying stent 82 as further described below. This exchange is illustrated in FIGS. 3E and 3F.

However, it should be appreciated that such an exchange need not occur. Rather, the original guidewire device inside the balloon catheter (or any other catheter used) may be that of item 80, instead of the standard guidewire 70 shown in FIG. 4A. Thus, the steps depicted in FIGS. 3E and 3F (hence, the figures also) may be omitted.

In addition, there may be no use in performing the step in FIG. 3D of advancing the balloon catheter past the lesion, since such placement is merely for the purpose of avoiding disturbing the site of the lesion by moving a guidewire past the same. FIG. 3G illustrates the next act in either case. Particularly, the balloon catheter is withdrawn so that its distal end 76 clears the lesion. Preferably, delivery guide 80 is held stationary, in a stable position. After the balloon is pulled back, so is delivery device 80, positioning stent 82 where desired. Note, however, that simultaneous retraction may be undertaken, combining the acts depicted in FIGS. 3G and 3H. Whatever the case, it should also be appreciated that the coordinated movement will typically be achieved by virtue of skilled manipulation by a doctor viewing one or more radiopaque features associated with the stent or delivery system under medical imaging.

Once placement of the stent across from dilated segment 62′ is accomplished, stent deployment commences. The manner of deployment is elaborated upon below. Upon deployment, stent 82 assumes an at least partially expanded shape in apposition to the compressed plaque as shown in FIG. 31. Next, the aforementioned postdilatation may be effected as shown in FIG. 3J by positioning balloon 74 within stent 82 and expanding both. This procedure may further expand the stent, pushing it into adjacent plaque—helping to secure each.

Naturally, the balloon need not be reintroduced for postdilatation, but it may be preferred. Regardless, once the delivery device 80 and balloon catheter 72 are withdrawn as in FIG. 3K, the angioplasty and stenting procedure at the lesion in vessel 60 is complete. FIG. 3L shows a detailed view of the emplaced stent and the desired resultant product in the form of a supported, open vessel.

In the above description, a 300 cm extendable delivery system is envisioned. Alternatively, the system can be 190 cm to accommodate a rapid exchange of monorail type of balloon catheter as is commonly known in the art. Of course, other approaches may be employed as well.

Furthermore, other endpoints may be desired such as implanting an anchoring stent in a hollow tubular body organ, closing off an aneurysm, delivering a plurality of stents, etc. In performing any of a variety of these or other procedures, suitable modification will be made in the subject methodology. The procedure shown is depicted merely because it illustrates a preferred mode of practicing the subject invention, despite its potential for broader applicability.

A more detailed overview of the subject delivery systems is provided in FIG. 4. Here, a delivery system 100 is shown along with a stent 102 held in a collapsed configuration upon the delivery guide member. A tubular member 104 is provided over and around the stent to restrain it from expanding. The tubular member may fully surround the stent or only subtend a partial circumference of the stent, it may be split, splittable, comprise a plurality of members or be otherwise provided around the stent to hold or restrain it in a collapsed profile. Exemplary delivery systems are noted above.

In any case, the delivery guide preferably comprises a flexible atraumatic distal tip 106 of one variety or another. On the other end of the delivery device, a handle 108 is preferably provided.

The handle shown is adapted for rotable actuation by holding body 110, and turning wheel 112. Alternatively, or additionally, a slide or lever may be provided for delivery device actuation. The handle may also include a lock 114. Furthermore, a removable interface member 116 facilitates taking the handle off of the delivery system proximal end 118. The interface will be lockable with respect to the body and preferably includes internal features for disengaging the handle from the delivery guide. Once accomplished, it will be possible to attach or “dock” a secondary length of wire 120 on the delivery system proximal end, allowing the combination to serve as an “exchange length” guidewire, thereby facilitating changing-out the balloon catheter or performing another procedure. Alternatively, the wire may be an exchange-length wire.

FIG. 4 also shows packaging 150 containing at least one coiled-up delivery systems 100. When a plurality of such systems are provided (in one package or by way of a number of packages held in stock), they are typically configured in support of a methodology where an appropriate one is picked to reach a target site and deploy a stent without unintended axial movement of the same as per the methodology of Ser. No. 10/792,684, referenced above. Thus, the packaging may serve the purpose of providing a kit or panel of differently configured delivery devices. In the alternative, the packaging may be configured as a tray kit for a single one of the delivery systems.

Either way, packaging may include one or more of an outer box 152 and one or more inner trays 154, 156 with peel-away coverings as is customary in packaging of disposable products provided for operating room use. Naturally, instructions for use 158 can be provided therein. Such instructions may be printed product or be provided in connection with another readable (including computer-readable) medium. The instructions may include provision for basic operation of the subject devices and associated methodology.

Regarding the specifics of the delivery device, it may be provided as in any of the above-referenced patent filings or otherwise. It preferably is one that maintains a constant size over its length during, or after, deployment of the stent. In regard to any delivery system employed, it is to be understood that conventional materials and techniques may be employed in the system construction. In this regard, it will often be desired to provide a lubricious coating or cover between moving components to reduce internal system friction.

In addition, it is to be understood that various radiopaque markers or features may be employed in the system to 1) locate stent position and length, 2) indicate device actuation and stent delivery and/or 3) locate the distal end of the delivery guide. As such, various platinum (or other radiopaque material) bands or other markers (such as tantalum plugs) may be variously incorporated into the system. Alternatively, or additionally, the stent stop or blocker member may be made of radiopaque material. Especially where the stent employed may shorten somewhat upon deployment, it may also be desired to align radiopaque features with the expected location (relative to the body of the guide member) of the stent upon deployment. For example, it may be desired to incorporate radiopaque features into the restraint and/or bridge or connector sections so that the deployment motion of the device is visible under fluoroscopy. Exemplary markers that may be of use are shown at a proximal end of the stent in FIG. 4 as elements A and A′—on the delivery guide body and tubular member, respectively—and at a distal end of the stent on the restraint as element B.

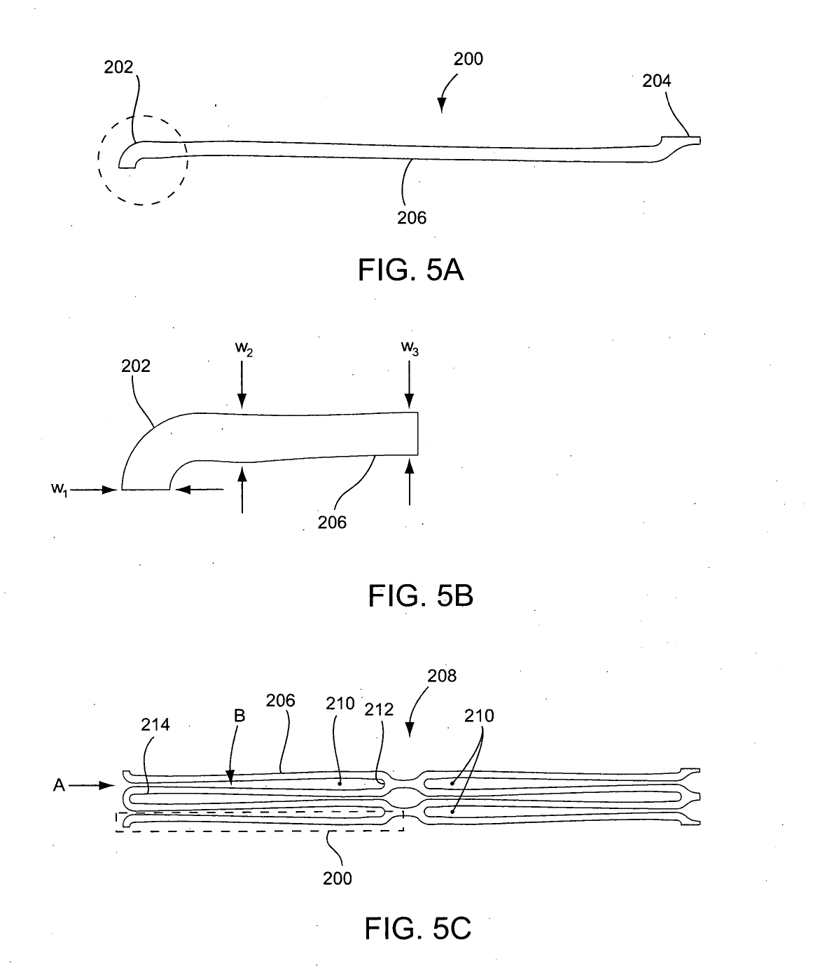

Returning now to the stent configurations of the present invention, FIGS. 5A-5C provide further details regarding the stent pattern introduced in FIGS. 2A and 2B. FIG. 5A shows a stent strut section 200 according to that variation of the invention, but in a precursor or fully compressed state. The strut is shown with end 202 and bridge section portions 204 provided between circumferentially adjacent struts.

The strut portions may have different width sections W₁, W₂ and W₃, where W₁>W₂>W₃ as shown in FIG. 5B. The additional bulk of material in these regions helps to minimize stresses/stains in the corresponding regions where curvature results in an increase or concentration of stresses. A medial section 206 of the strut will generally experience the lowest bending stresses. As such, material removal along at least a portion of the length inward of the end sections of the strut will be acceptable, even desirable.

Turning now to FIG. 5C, a stent section 208 is shown assembled from several of strut sections 200 (one such element as presented in FIG. 5A highlighted in the dashed box in FIG. 5C) including the medial stent strut body 206 and end and junction portions 204 and 206. In this view, the manner in which the constituent parts of the strut and end features interact upon compression to form teardrop-shaped openings 210 is clearly visible. The profiles are generated by and between radius section 212, point 214 and inner edge of the medial strut section 206. What is more, the struts themselves form a closely-packed series of teardrop shaped forms.

As referenced above, the medial strut sections 206 may or may not contact. Thus they may be parallel to one another at the “tip” of the teardrop shape. If they do contact, the point of contact may vary from point A adjacent other strut connection sections to a more medial section of the stent at point B. In any case, such variations are contemplated within the scope of the invention as are various ratios of widths and thicknesses of the stent struts.

In such stent designs, strut width may be between about 0.002 and about 0.005 inches and strut thickness from about 50% to about 150% that of the width. Preferably, the radius between adjacent struts is a full radius, meaning that it offers a smooth rounded transition between the strut adjacent sections. While not necessarily circular, the form will generally have an effective or average radius of between about 50% to about 200% adjacent strut width. For a stent of the noted sizes, the radius will generally be no less than about 0.001 inches, nor need it be greater than about 0.005 inches—though variation from these exemplary dimensions is within in the scope of the present invention.

As evident from observation of the design, it maximizes compaction capability and minimizes the material employed. The struts are preferably collapsed as shown to a generally smooth or straight profile without and (or at least any significant) stress-raising discontinuities or changes in curvature. The compressed configuration offers a highly organized and symmetrical packing of elements. When the stent is thus-compacted, expansion capability is maximized in that bending forces on the stent are widely distributed, generally without contact between members which can result in highly localized stresses.

As with other stents, the stent described above is typical in its use of superelastic NiTi or NITINOL material. Still, in instances where another material is to be used, the same production methodology may be employed. However, its shape will differ (at least in terms of its uncompressed shape) in order to achieve the desired compressed shape described above. Note, however, that the same process may be advantageously used to attain compressed stent shapes or shapes for other prostheses as desired.

However, even when a different material is used for producing a compressed stent shaped like that shown in FIGS. 5A-5C, the uncompressed state of the design will be expected to display a variant of an S-curve. But, it will not be a curve identical to that produced employing the noted material. Indeed, due to the subtle variance and difficulty describing such shapes it is also noted that the inventors hereof reserve the right to claim the particular shapes of the compressed and uncompressed stent as shown in FIGS. 2A/2B and 5A/5B in reference to these figures as well as site the figures.

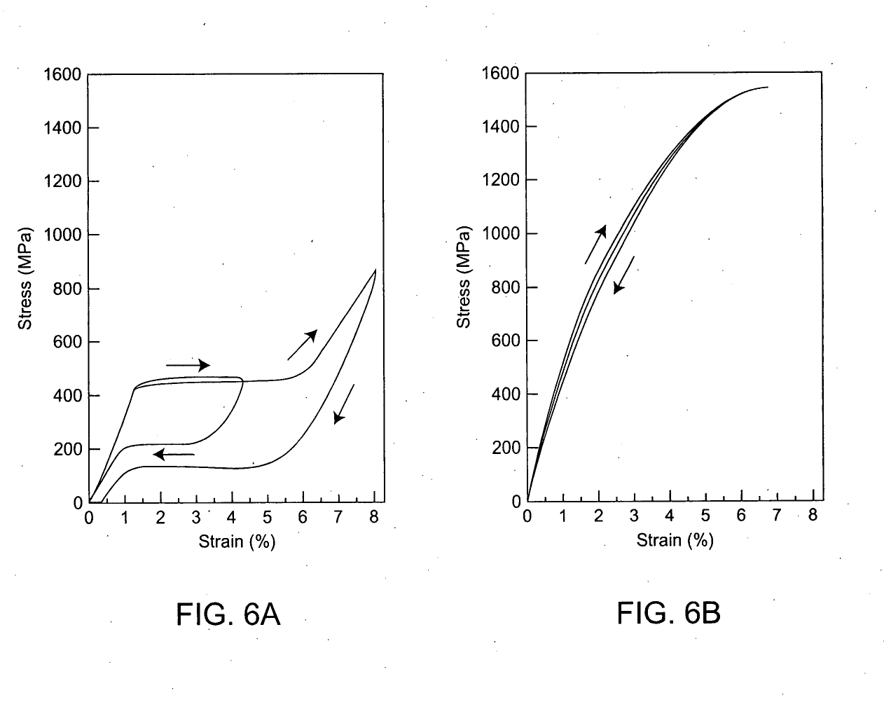

Another class of stents according to the present invention can be produced employing cold worked or pseudoelastic nickel-titanium alloy. One example of such alloy is the FHP-NT from Furukawa; another is described in U.S. Pat. No. 6,602,272, which is incorporated herein by reference. As background, FIGS. 6A and 6B provide stress-strain curves illustrating typical NITINOL performance and FHP-NT alloy performance, respectively. The superelastic NiTi alloy performance in FIG. 6A exhibits the typical “flag” shaped profile. In this, at a strain rate of less than about 1.5%, the stress/stain curve flattens as the material transforms from an austenitic state to a martinsitic state. Such action facilitates the hinging noted above. However, it also limits the stiffness of the material in the pseudoelastic range “P” of the material behavior.

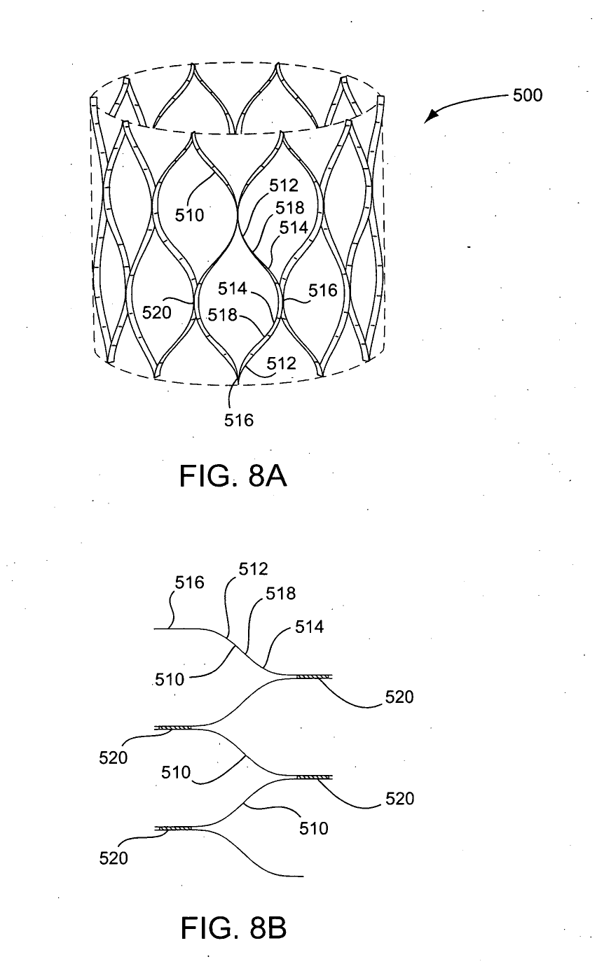

In contrast, the FHP-NT material of which the performance curve is shown in FIG. 8B displays no plateau region. However, it is can be reversibly deformed at similar strain rates to superelastic NITINOL. Where the FHP-NT type material offers an advantage, however, is in that it behaves in a substantially “linear” fashion over about 450 Mpa stress. Hence, it can be employed as described above in defining a different approach to stent sizing and preloading approaches. Furthermore, substantially less metal may be employed in reaching comparable stent radial forces. In addition, it may be possible to apply stents made of FHP-NT type material in new applications because of the greatly widened window of forcing possibilities and force-tuning offered through use of the subject material.

In one manner of using this material for stent production, it is contemplated to employ an FHP-NT type material stent a stress levels of at least about 1.5% upon implantation. With FHP-NT, in-situ strain rates up to about 4% or more (including intermediate values) are further possible. By—in effect—making the material work harder, less material or higher forces or a combination of the same may be achieved with a given stent.

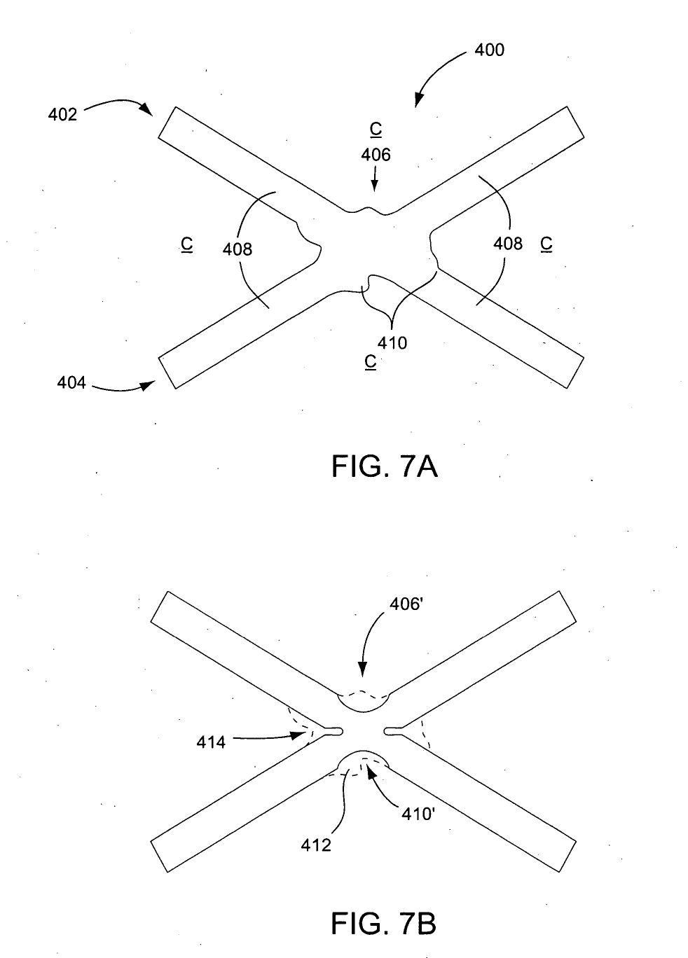

As for the configuration of the stent itself, a number of approaches to stent construction may be employed with the subject material. FIG. 7A shows a plan view of a stent section 400 that is part of a greater whole. Here, ribbons or wires 402 and 404 are joined at a welded strut junction 406 and may be employed in providing and FHP-NT type stent. Stent cells “C” between struts or arms 408 are defined by weaving or overlapping the crossing wires 402, 404 that are then connected at their respective junctions to form the plurality of strut or arm sections. At least when friction welding is employed, the welding process may result in slag material 410 that flows outward as the vibrating material flows and fuses.

While other connection approaches may be desirable, friction welding may be preferred in view of the size and strength of the bond formed as well as the final part thickness obtained which is less than the original thickness of the overlapping section of material.

In any case, as shown in FIG. 7B, a new machined junction profile 406′ is shown in solid line. The broken line illustrates the unwanted weld slag 410′ and stress-concentrating regions that is removed by laser machining, EDM, etc. in providing a final strut junction shape. The clean-up procedure thereby provides uniform radius sections 412 and stress reliever features 414 between adjacent struts. Such a profile yields a highly functional geometry that produces more uniform loading of the stent under large deformations associated with restraint on a delivery device as well as the desired in-situ strains that figure into fatigue life. In addition to such clean-up, additional modifications to the junction or struts themselves may be undertaken to improve stent performance—possibly in line with the teaching expressed above.

Whereas this mode of stent construction is especially desirable where the ratios of material width to thickness are about 1: 1 or the material width is greater than the thickness, another mode of stent construction may be preferable where the material depth or radial thickness is substantially greater than its width. Specifically, this mode is that disclosed in U.S. Pat. No. 6,454,795, except made of a material as disclosed herein.

In regard to the stent design, however, stent 500 shown in FIG. 8A comprises a repeating element of each strut arm or limb 510 of the stent that has two curves 512 and 514 of substantially equal radius, substantially equal length and opposite direction. The short straight segments 516 at the ends of each strut element are shown parallel to one another. A mid-strut portion 518 lies between the two curved segments of each repeating element of the stent strut. Depending on the overall length of the stent, the same piece of wire may bend back and forth in a sinusoid wave, to form a series of strut elements 510 along the length of the stent.

The short straight segments 516 of struts or limbs are joined, either by welding, soldering, riveting, or gluing, as depicted in FIG. 8B or otherwise. A plurality of identical strut elements are joined in this way to form a substantially cylindrical structure the exterior of which is shown in phantom line in FIG. 8A.

The number of strut or limb elements in each length of wire can be varied according to ratio of length to width required for the specific application in which the stent is to be employed. The radius and length of the curves 512 and 514 can be altered to effect the orientation of the section of the limb element that lies between the curves, the mid-section 518. In addition mid-section 518 may vary in length as may be required for the particular application. Still further, other embodiments of FHP-NT type stents may be constructed as well. The two species provided are merely intended to support a genus of such stents. The same is intended for the genus of materials as employed to stent construction.

The invention further includes methods that may be performed using the subject devices or by other means. The methods may all comprise the act of providing a suitable device. Such provision may be performed by the end user. In other words, the “providing” (e.g., a delivery system) merely requires the end user obtain, access, approach, position, set-up, activate, power-up or otherwise act to provide the requisite device in the subject method. Methods recited herein may be carried out in any order of the recited events which is logically possible, as well as in the recited order of events.

Exemplary aspects of the invention, together with details regarding material selection and manufacture have been set forth above. As for other details of the present invention, these may be appreciated in connection with the above-referenced patents and publications as well as generally know or appreciated by those with skill in the art. For example, one with skill in the art will appreciate that a lubricious coating (e.g., hydrophilic polymers such as polyvinylpyrrolidone-based compositions, fluoropolymers such as tetrafluoroethylene, hydrophilic gel or silicones) may be placed on the core member of the device, if desired to facilitate low friction manipulation. The same may hold true with respect to method-based aspects of the invention in terms of additional acts as commonly or logically employed.

In addition, though the invention has been described in reference to several examples, optionally incorporating various features, the invention is not to be limited to that which is described or indicated as contemplated with respect to each variation of the invention. Various changes may be made to the invention described and equivalents (whether recited herein or not included for the sake of some brevity) may be substituted without departing from the true spirit and scope of the invention. In addition, where a range of values is provided, it is understood that every intervening value, between the upper and lower limit of that range and any other stated or intervening value in that stated range is encompassed within the invention.

Also, it is contemplated that any optional feature of the inventive variations described may be set forth and claimed independently, or in combination with any one or more of the features described herein. Reference to a singular item, includes the possibility that there are plural of the same items present. More specifically, as used herein and in the appended claims, the singular forms “a,” “an,” “said,” and “the” include plural referents unless the specifically stated otherwise. In other words, use of the articles allow for “at least one” of the subject item in the description above as well as the claims below. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

Without the use of such exclusive terminology, the term “comprising” in the claims shall allow for the inclusion of any additional element—irrespective of whether a given number of elements are enumerated in the claim, or the addition of a feature could be regarded as transforming the nature of an element set forth n the claims. Except as specifically defined herein, all technical and scientific terms used herein are to be given as broad a commonly understood meaning as possible while maintaining claim validity.

### CLAIMS

The breadth of the present invention is not to be limited to the examples provided and/or the subject specification, but rather only by the scope of the claim language. That being said, we claim:

1. A self-expanding stent comprising a plurality of struts, each strut having two ends, each strut end connected to a circumferentially adjacent strut end, the stent having an expanded shape and a compressed shape, wherein in the compressed shape, the struts define a plurality of teardrop-shaped openings along substantially a whole length of the struts.

2. A self-expanding stent comprising a plurality of struts, each strut having two ends, each strut end connected to a circumferentially adjacent strut end, the stent having an expanded shape and a compressed shape, wherein in the compressed shape, the struts define a plurality of teardrop-shaped openings only.

3. A self-expanding stent comprising a plurality of struts, each strut having two ends, each strut end connected to a circumferentially adjacent strut end, the stent having an expanded shape and a compressed shape, wherein in the compressed shape, the struts define a plurality of close-packed teardrop shaped forms.

4. A self-expanding stent comprising a plurality of struts, a medial portion of the struts having an S-curve shape as originally cut from a tube.

5. The stent of claim 4, wherein the tube has a diameter substantially equal to a fully-expanded shape of the stent.

6. The stent of claim 4, wherein the stent is adapted so that the struts are substantially straight along a medial section upon full compression of the stent.

7. The stent of claim 4, wherein the stent is adapted so at least a plurality of the struts define teardrop shaped forms upon full compression of the stent.

8. A method of making a self-expanding stent comprising:
providing a tube of elastic or superelastic material; and
cutting the tube in a pattern comprising a plurality of struts, a medial portion of the struts having an S-curve shape.

9. The method of claim 8, wherein the S-curve shape is defined so that in a compressed state the struts define forms selected from:
teardrop-shaped openings along substantially a whole length of the struts, teardrop-shaped openings only, a plurality of close-packed teardrop bodies, and substantially straight strut medial sections.

10. A stent made according to the method of claim 8 or 9.

11. The stent of any of claims 1-7 and 10, wherein each strut comprises a medial section and two end sections, wherein the medial section has a first width and the end sections have a second width greater than the first width.

12. The stent of any of claims 1-7 and 10, wherein the struts define a plurality of closed cells, wherein four struts defining each of the closed cells.

13. The stent of claim 12, wherein a necked down bridge section is provided between axially adjacent struts

14. The stent of claim, wherein terminal ends of the cells are rounded-off so as to be atraumatic.

15. A method of designing a self-expanding stent, the method comprising:
providing a precursor stent strut design in a desired compressed configuration;
expanding the precursor stent strut design to a desired expanded configuration; and
setting a stent strut cutting pattern for the self-expanding stent corresponding to the expanded configuration shape of the precursor stent design.

16. The method of claim 15, wherein the precursor stent strut design is part of a physical stent and the expanding is by physical expansion.

17. A self-expanding stent made by a process comprising:
providing a stent design as described in claim 15; and
cutting a stent pattern in tubing according to the stent design.

18. A self-expanding stent made of a metal suitable for implantation in a mammalian body at stress levels of at least about 1.5%.

19. The stent of claim 18, wherein the material comprises an FHP-NT type material.

20. The stent of claim 18, wherein the stent comprises cells comprising crossing wires, wherein the wires connect at junctions to form a plurality of strut sections, wherein uniform radius sections are provided between adjacent struts.

21. The stent of claim 20, wherein the wires have a smaller radial thickness than width.

22. The stent of claim 18, wherein the stent comprises cells comprising crossing wires, wherein the wires connect at junctions to form a plurality of struts, wherein the struts are curved in opposite directions in an expanded configuration.

23. The stent of claim 22, wherein the wires have a larger radial thickness than radial width.

24. A self-expanding stent comprising:
crossing wires connected at junctions to form a plurality of strut sections,
wherein uniform radiused sections are provided between adjacent struts.

25. The stent of claim 24, wherein the stent comprises an FHP-NT type material.

26. The stent of claim 20 or 24, wherein the wires are connected by welding.

27. The stent of claim 26, wherein the welding is friction welding.

28. The stent of claim 20 or 24, comprising a plurality of closed cells.

29. The stent of claim 28, wherein four struts define the closed cells.

30. A self-expanding stent comprising a plurality of struts, each strut having two ends, each strut end connected to a circumferentially adjacent strut end, the stent having an expanded shape and a compressed shape, the improvement consisting of: the struts defining a plurality of teardrop shapes in a compressed configuration.

31. The stent of claim 30, wherein the teardrop shapes are formed over substantially an entire length of the struts.

32. The stent of claim 30, wherein the struts define a plurality of teardrop-shapes only.

33. The stent of claim 30, wherein the tear-drop shapes are closely packed.
